(19) United States
(12) Patent Application Publication
(10) Pub. No.: US 2008/0212024 A1
(43) Pub. Date: Sep. 4, 2008

(54) CUSTOMIZED CONTACT LENSES FOR REDUCING ABERRATIONS OF THE EYE

(76) Inventor: Shui T. Lai, Encinitas, CA (US)

Correspondence Address: SF Bay Area Patents, LLC;Attn: Andrew V. Smith, Ph.D., 601 Van Ness Avenue, #1108, San Francisco, CA, 94102, US

(21) Appl. No.: 11/857,365

(22) Filed: Sep. 18, 2007

Related U.S. Application Data
(60) Provisional application No. 60/826,043, filed on Sep. 18, 2006.

(51) Int. Cl. A61B 3/10; G02C 7/04; G02C 7/16

(52) U.S. Cl. 351/205; 351/177; 351/160 R; 351/162

(57) ABSTRACT

Higher order aberrations of an eye are reduced. Wavefront aberrations of the eye are measured, providing wavefront information and/or a wavefront map. A mask is derived from the wavefront information and/or wavefront map based on the measured wavefront aberrations. A customized contact lens is formed including the mask. The deriving of the mask includes optimizing a figure of merit using a PSF, a MTF, and/or another image quantifier.

### PRIORITY

This application claims the benefit of priority to United States provisional patent application no. 60/826,043, filed Sep. 18, 2006, which is incorporated by reference.

### BACKGROUND

If one looks at the eye as an optics system, one may be surprised at how poor the optical quality is, comparing it to a piece of an off-the-shelf optic such as a spherical lens. One way to characterize these aberrations is using Zernike polynomials. The term “higher order” refers to the third and higher order terms of Zernike polynomials that are utilized for more fully characterizing aberrations of the eye. The second order terms include defocus, astigmatism and axis, which are the lower order terms which are typically correctible with conventional spectacle eyeglasses. When an eye has a keratoconus condition, or has encountered traumatic damages, such as acid burns, or has undergone corneal transplantation surgery, the aberrations are much more severe and the quality of vision is significantly deteriorated. It is advantageous to provide a method of improving quality of vision in these highly distorted eyes. Even for non-diseased, normal eyes, the percentage of eyes that can provide a best corrected vision of 20/15 or better vision is less than 35%. During nighttime, or in dark viewing conditions, headlight glares, and halos can be debilitating for many normal sighted people. Hence it is desirable to improve vision for the normal or average eyes.

Previously, various patents have attempted to improve vision by restricting the aperture of the eye by having a pin hole on a contact lens. This provides improvement in the depth of field, but it basically eliminates the focus of the cornea. In addition, the spatial resolution is poor. Legerton et. al., in U.S. Pat. No. 5,662,706, which is incorporated by reference, have tried to remedy this drawback by proposing a larger pin hole in the center, by having a ring shape occlusion on a contact lens. The position of the ring and the size of the ring are arbitrary, and key determinant of its inner diameter is “to maintain retinal illumination”.

It is recognized in the present invention that the most dominant aberrations are those of the lower orders in the Zernike representation. For example, sphere and cylinder are represented by the second order terms, and they have the majority portion of most eye aberrations, in the range of 60 to 90%, depending on the individual. The next largest is the spherical aberration, Z(4,0) term. Then the third order terms, the rest of the 4^{th} order terms, etc. Even through an aberrated wavefront, the optics of the eye can be decomposed into Zernike terms, each term being only a building block of the entire aberration, which is three dimensional including x-y spatial distribution of the optical path difference (OPD) in z-amplitude. It is further observed that by aperturing the pupil of the eye, one can theoretically calculate the wavefront of the eye at a reduced aperture, which is typically smaller in terms of peak-to-valley in normally sighted eyes. However, it is not the case, for example in the case of a keratoconus eye. Therefore, arbitrarily aperturing the pupil will not produce predictable improved quality in vision.

### SUMMARY OF THE INVENTION

A system is provided for reducing wavefront aberrations of an eye. A wavefront aberrometer measures wavefront information of an eye. A computer program derives a mask covering a wavefront aberration portion of the the eye based on the measured wavefront information. A machine forms a customized lens including the mask.

The deriving of the mask may include an optimization of a figure of merit using a PSF, a MTF, another image quantifier, or combinations thereof. The figure of merit may include a light intensity volume within a circle centered at a PSF peak or at a geometric mean of a PSF intensity distribution. The figure or merit may use MTF, for example, area under a MTF curve, Nyquist limits, neural response as a function of spatial frequency, best sphero-cylindrical refraction or best calculated eyeglasses prescription, or combinations thereof.

The customized contact, intra-corneal lens or intraocular lens may be configured to reduce the wavefront aberrations to make at least one additional line readable in an EDTRS or Snellen eye chart or both. The lens may be configured to reduce the wavefront aberrations to improve contrast sensitivity by at least 0.15 in log scale or 40% in linear scale, or both.

The machine may write the customized mask accounting for a de-centered position of the contact lens on the eye.

The mask may be shaped to block distorted rays from one or more distortion regions of the eye. The mask may include one or more masked regions from a perimeter of the lens or a ring on the lens or a combination thereof.

A method of reducing higher order aberrations of an eye is also provided. Wavefront aberrations of an eye are measured. A wavefront map or other wavefront information, or both, is/are generated based on the measuring. A mask is derived from the wavefront map or other wavefront information, or both. A customized contact lens is formed including the mask.

The deriving of the mask may include optimizing a figure of merit using a PSF, a MTF, another image quantifier, or combinations thereof. The figure of merit may include a volume of light rays within a circle centered at a PSF peak or at a geometric mean of a PSF intensity distribution. The figure or merit may use MTF, e.g., an area under MTF curve, Nyquist limits, neural response as a function of spatial frequency, best sphero-cylindrical refraction or best calculated eyeglasses prescription, or combinations thereof.

The wavefront aberrations may be reduced to make at least one additional line readable in an EDTRS or Snellen eye chart or both, and/or contrast sensitivity may be improved by 0.15 or more in log scale and/or 40% or more in linear scale.

The customized mask may be written while accounting for a de-centered position of the contact lens on the eye. The mask may be shaped to block distorted rays from one or more regions of the eye. One or more masked regions may be formed at a perimeter of the lens or a ring on the lens or a combination thereof.

A contact lens is also provided to improve the quality of vision of an eye after pupil dilation. A masked region is provided for the lens that substantially blocks rays from transmitting through the lens and pupil, except at a central region.

The diameter of the contact lens may cover the entire dilated pupil and/or the masked region may include the entire dilated pupil region.

The central region transmitting light rays may include an area having a dimension between approximately 2 mm and 4 mm. The central region may include a tinted region that partially transmits light rays. The tinted region may include substantially the entire central region.

The contact lens may have a refractive power in a range from −3 to +3 diopters.

The central region of the mask may also include one or more specific mask portions determined based on a measurement of wavefront aberrations of an eye, and generation based on the measurement of a wavefront map or other wavefront information, or both.

### BRIEF DESCRIPTION OF THE DRAWINGS

FIG. 1 is a block diagram illustrating certain embodiments.

FIG. 2(A)-2(G) schematically illustrate customized lenses in accordance with several embodiments.

FIGS. 3(A)-3(D) schematically illustrate effects of a keratoconus condition of an eye.

FIGS. 4(A)-4(C) schematically illustrate improved quality of vision for a kerotoconus eye improved with a customized lens in accordance with certain embodiments.

FIGS. 5(A)-5(C) schematically illustrate improved quality of vision for a keratoconus eye improved with a customized lens in accordance with another embodiment.

### DETAILED DESCRIPTION OF THE EMBODIMENTS

Techniques and embodiments are described below wherein wavefront aberrations of the eye are reduced. Wavefront aberrations of the eye are first measured using a wavefront aberrometer, which is for example commercially available from Ophthonix, Wavefront Sciences, Visx, or Bausch and Lomb.

### QUALITY OF VISION METRIC

The quality of vision can be assessed using this wavefront (WF) information or the WF map. A number of quantifiers can be used as a measure of quality of vision. Point Spread Function (PSF) of the WF map is a good measure. The tightness of the light distribution of a point image indicates the “goodness” of the optics of the eye.

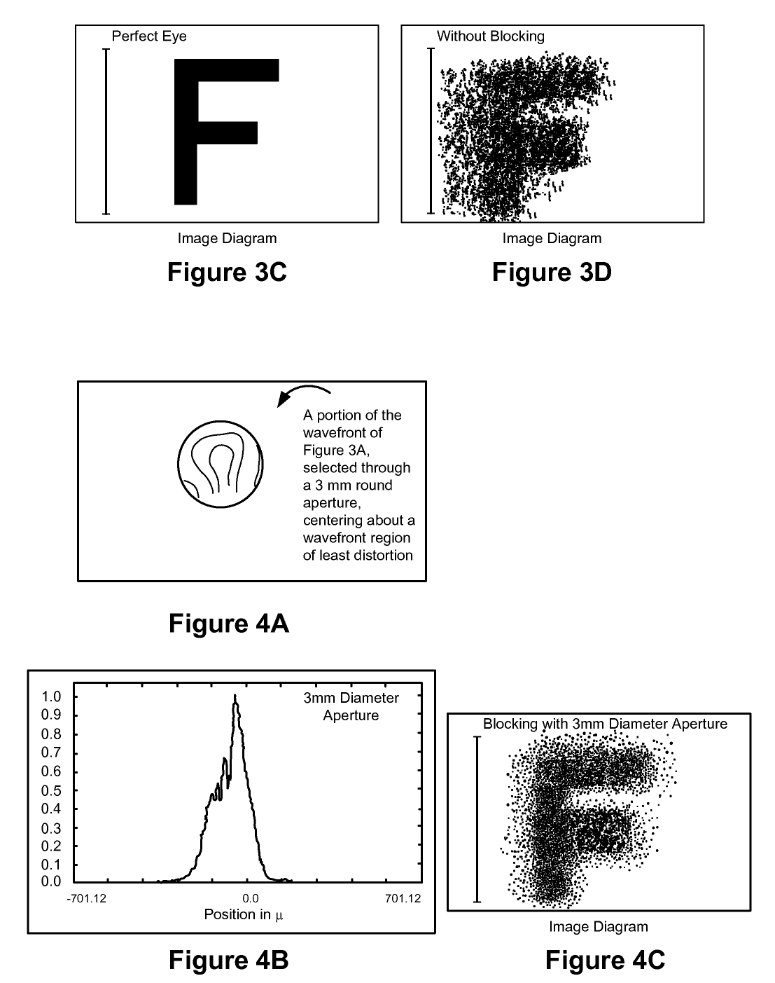

A figure of merit or a quality of vision metric can be constructed as illustrated in the following. In one embodiment, it is a quantity relating to the tightness of the PSF, an area of a circle centered at the peak of the PSF that encircles 85% of the light intensity. The 85% value is arbitrary, other values can also can be used as a measure of the tightness of the PSF. Other variations may include, but are not limited to, putting the center of the circular zone at the geometric mean of the intensity distribution. In another embodiment, the Modulation Transfer Function (MTF) is a quantitative measure for the optical quality of the eye. It provides a theoretical contrast sensitivity threshold level of the eye function at various spatial frequencies. Again, there can be a number of methods to construct a figure of merit using the MTF. One method used the area under the MTF curve. The larger the integrated area, the better the quality of vision is deemed to be in this case. An improved method using MTF is to include the effects of the eye's Nyquist's limits due to the sampling limit of the retina, thereby the area under the MTF is limited to the Nyquist's frequency. In another improvement, a neural response as a function of the spatial frequency is superimposed over the MTF to accurately reflect a functional MTF. There are other methods to define the quality of vision metric. The best sphero-cylindrical refraction, or the best calculated eyeglasses prescription may be predicted based on a wavefront (WF) map of an eye and its figure of merit of various quantifiers for quality of vision. A WF map may be illustrated by two dimensional representations such as the examples of FIGS. 3A, 4A and 5A. In those examples, contour lines are provided according to a height profile or optical path difference (OPD) profile of an eye. Peaks in the WF maps represent OPD profile maxima near cones of the eye. Certain metrics (or quantifiers) have been proposed in this regard by Ray Applegate, David Williams, or Larry Thibos, particularly in Optometry and Vision Science, Volume 80, No. 1, 2003, pp 36-42, in the Journal of Vision, Volume 4, 2004, pp. 322-328, and in the Journal of Vision, volume 4, 2004, pp. 310-321, which are fully incorporated by reference.

Based on the wavefront (WF) map, a figure of merit is calculated using SPF, MTF, or other applicable quantifiers of quality of vision or quality of vision metric. A customized mask is designed for such WF map to provide an improved figure of merit for the eye. It is preferable to achieve an improvement that can result in an increase of one or more the following objectives: increased visual acuity, increased contrast sensitivity, and increased MTF figure of merit, particularly at functional frequencies. In embodiments described herein, improvement of one line or more in reading an EDTRS or Snellen eye chart, and/or improvement in contrast sensitivity of at least 0.15 in log scale (40% improvement in a linear scale) is achieved. Such a mask design is incorporated in a contact lens, intracorneal lens or an intraocular lens, which is to be implanted in the eye.

The orientation and the location of the mask is relevant to the orientation and location of the aberration map of the eye. Ideally, the mask provides maximum benefits when the mask is located and orientated at the intended location overlapping the WF aberrations of the eye. However, there is a great advantage using this method over conventional methods of correcting higher order aberrations, in which the wavefront errors are corrected by compensating the Optical Path Difference (OPD). For example, in a WF-guided LASIK, a corneal tissue profile is removed or ablated by a laser. The ablated tissue profile is designed to have an OPD distribution opposite to the OPD map of the eye before surgery. Likewise, in the WF-guided lenses proposed by Ophthonix, the index of refraction in the proprietary material inside the lens is modified to cancel the WF error map of the eye. This is would be ideal if the laser ablation is exactly precise, and no errors are made as a result of eye movement during the entire laser procedure (see, e.g., U.S. Pat. No. 7,220,255 by the same inventor as the present invention, which is incorporated by reference). Also, the healing process after the surgery would not cause any change that may wipe out the effect of intended correction profile. All of these are difficult to achieve and the healing process particularly is not predictable in conventional techniques. In the case of WF-guide eyeglasses, the eye movement at various glazing angle limits the benefits of such design. Slight misalignment due to misplacement of the ablation profile in the case of LASIK, or misalignment of the WF spectacle lens or eye glazing at off axis angles, is detrimental to the quality of vision in conventional techniques. The alignment has to be accurate to within 0.5 mm. Even at 1 mm error mark, the benefit can be all but eliminated, or worse, can induce worse aberration than a surgery or a spectacle correction without attempting wavefront correction.

In a masked contact lens according to embodiments described herein, a small amount of contact lens movement is expected, particularly right after a blinking of the eye. However, the displacement is small and is in the order of about 1 mm or less. The reduction of benefits due to such shifting of the mask in a 1 mm displacement is expected to be minimal, or non-noticeable, as compared to a severe loss of benefits in the case of using a conventional wavefront profile cancellation method. One can estimate the reduction of benefit by calculating a change of unmasked area due to contact lens movement.

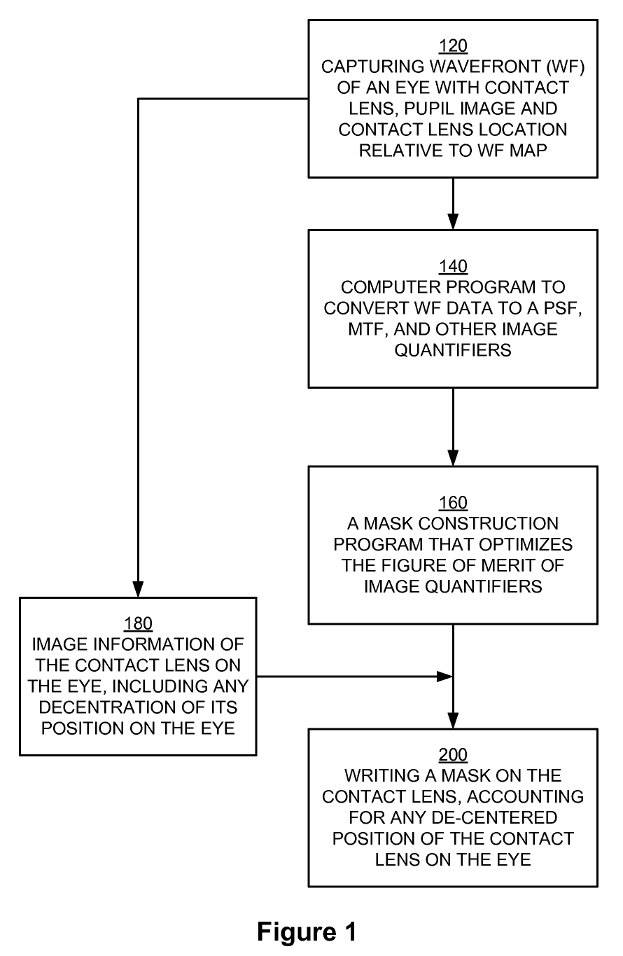

A method of improving quality of vision in aberrated eyes in accordance with a certain embodiments is shown schematically in FIG. 1. In box 120, a trial contact lens is put on the eye, and the WF profile of an eye including the contact lens is captured using a WF aberrometer. The pupil size and the location of the contact lens on the cornea are also captured.

In box 140, a computer program converts the WF map of the eye/contact lens into a figure of merit which is derived from a definition (or a metric) of using various image quality quantifiers. Examples have been described above and others are provided in references incorporated by reference. Among them are the PSF, MTF, OTF or Strehl's intensity ratio, all of which can be derived from the WF map.

The next step 160 is a computer program generating a mask profile over a contact lens, intra-corneal lens, or an intraocular lens. When this mask is applied over the WF map of the unmasked eye, the figure of merit of the selected image quality quantifier changes, and the visual acuity and contrast sensitivity is checked at that value of the improved figure of merit. A search loop is included in the program including one or more search algorithms. This enables the search to improve the figure of merit, which in turn, improves the spatial resolution, contrast sensitivity, and/or visual acuity. By way of example, if the spatial resolution of the eye as indicated by EDTRS chart test is not better than 20/25 without a mask, the outcome of using a mask generated by the search program shall provide a better than 20/25 visual acuity, and the final mask improves vision by at least one line in the eye chart, to 20/20 or better. Increased improvement of quality of vision is achieved through optimization of the mask profile.

In box 180, the pupil diameter and the location information of the contact lens on the cornea are extracted and input for mask making at box 200. The information may be obtained in conjunction with the measurements in box 120, when the WF information is being captured, and measured separately. The writing of the mask 200 can be achieved by a number of methods. One may use a direct printing method with permanent ink to fill the mask area. In another embodiment, a gradual transition of optical attenuation (or the degree of light transmission) can be added to the boundary of the mask, to avoid a sharp edge effect that may otherwise cause wave diffraction and thereby reduce contrast. The transition region or the border zone at the mask boundary may be in the range of 0.5 mm to 2.0 mm.

Alternatively, the mask can be made by imaging UV or other wavelengths light onto the contact lens surface, on which a light sensitive agent has been applied as a coating or soaked or absorbed into the lens. The light source will act upon this agent and change its transmission coefficient to form a mask of desired shape at the intended location of the lens and with a predetermined spatial distribution of transmission profile, including a transition region. A laser beam can be used to activate the agent. Photolithography methods may be used to write the mask.

### EXAMPLES OF MASK TYPE

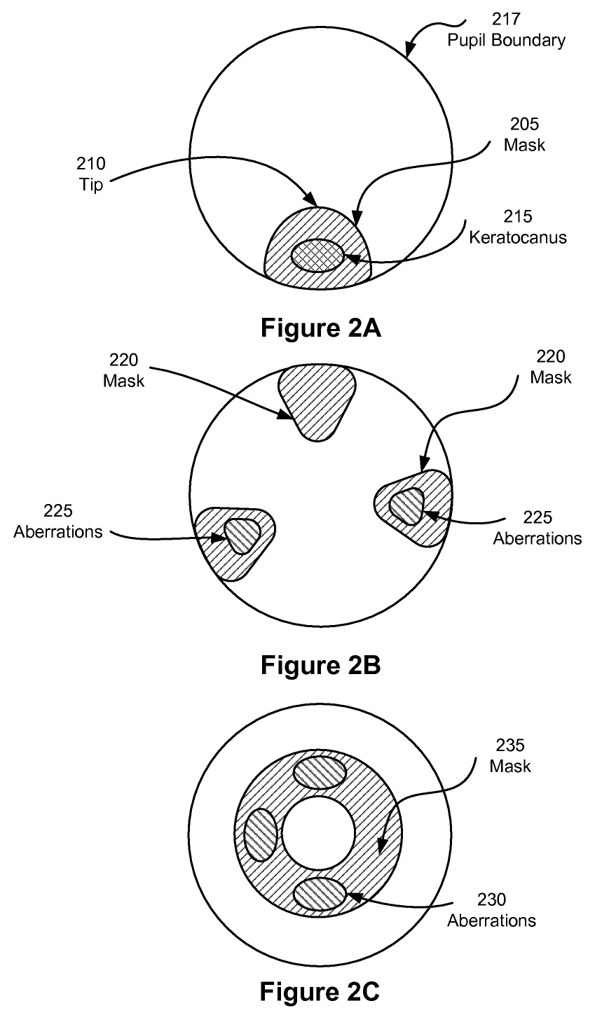

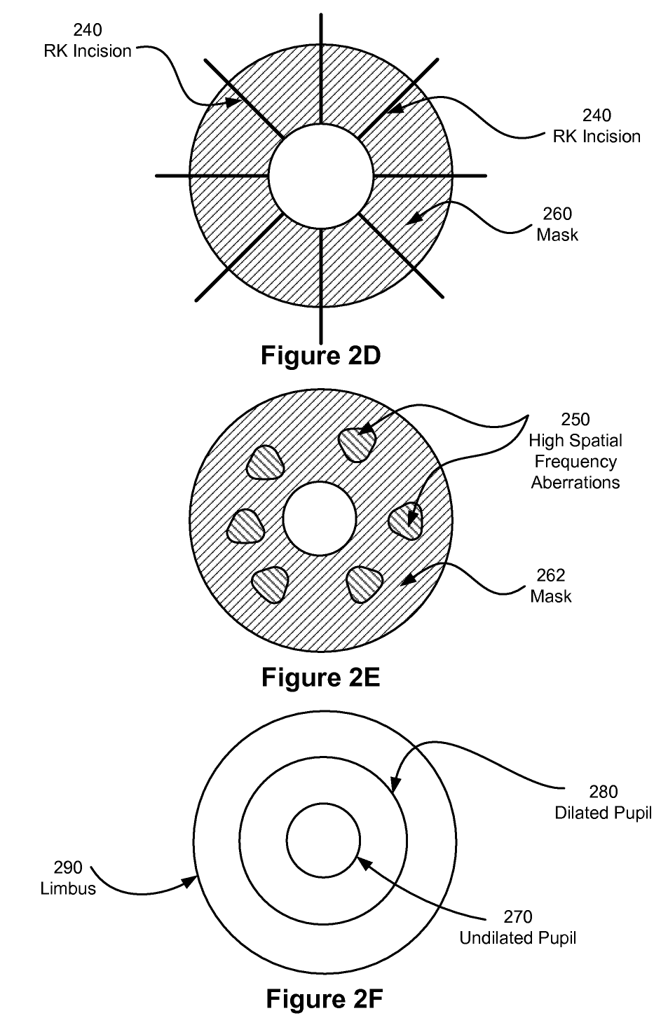

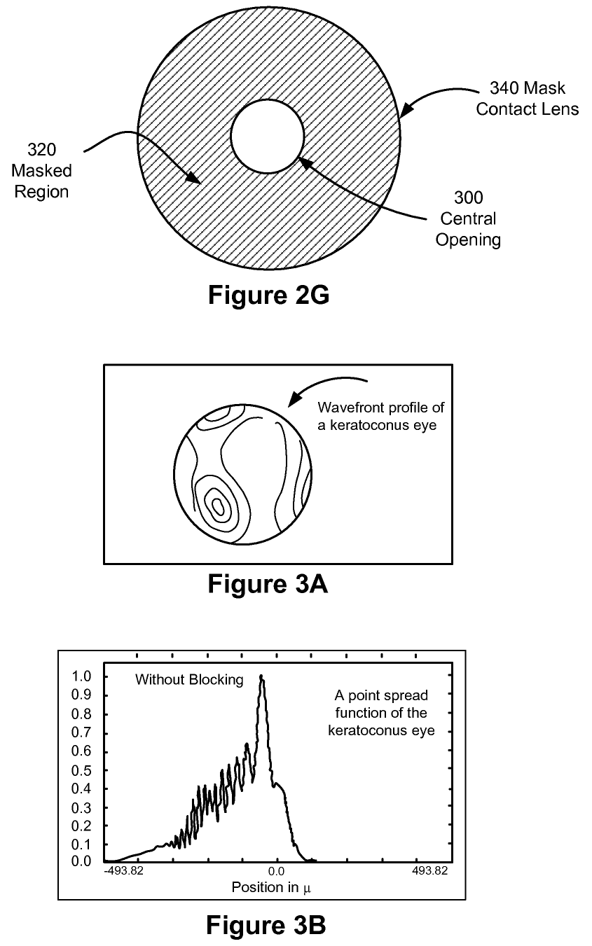

FIG. 2 schematically illustrates examples of various shapes of mask. In FIG. 2(A), a mask 205 is illustrated as covering a section of the cornea. The “tip” of a “pie-shaped mask” 210, does not have to be at or near the center of the eye, although it can be. For the case of a keratoconus condition with an inferior cone 215, the mask will substantially block distorted rays from the cone area. The pupil boundary is shown as 217 in FIG. 2(A), which is an non-limiting example. The boundary 217 may be, for example, at or near that of the dark pupil size or the largest natural pupil size of the eye. The contact lens that the mask is written on is covering substantially the entire pupil area of the eye extending to and covering part of the limbus of the eye (although not shown in FIG. 2(A).

In the following examples, the circular outer pupil boundary is shown similar to that in FIG. 2(A) as the pupil boundary of the eye. Multiple mask segments 220 at appropriate angular distribution are shown in FIG. 2(B), blocking distortions 225 from several peaks/or valleys in the OPD map of the eye (shown as darkened areas) near the boundary of the pupil diameter. These are advantageous for the cases that the eye aberrations are dominated by trefoil, or quatrefoil as represented by such Zernike terms. In FIG. 2(C), an eye aberration caused by multiple peaks or valleys 230 in the mid-central region is blocked out by a ring shaped mask 235. The inner and outer diameters of the “ring” are to be optimized with the specified WF profile of the eye. For distortions caused by Radial Keratotomy (RK) incisions 240 in FIG. 2(D), and for distortions with higher spatial frequency near the edge 250 of the pupil, like pentafoils and hexafoils or others in FIG. 2 (E), solid mask rings 260 and 262 covering the periphery region of the respective corneas may be applied. Again, the size of mask area is optimized not to result in a lower spatial resolution or lower contrast sensitivity in comparison to that of the unmasked eye.

### IMPROVING QUALITY OF VISION IN A DILATED EYE

Another application of a masked contact lens is for patients with normal vision undergoing pupil dilation examination. The aberrations of the eye increase quadratically with the pupil diameter, and the quality of vision after dilation typically experiences substantial decrease in quality of vision. Furthermore, the dilated pupil loses its ability to regulate the brightness level. The retinal image suffers both light intensity overload and a severe image blurring.

In one embodiment, the normal quality of vision is restored in dilated eyes using a masked contact lens covering substantially the dilated pupil area, preferably limbus to limbus. A schematic drawing of the eye is illustrated in FIG. 2(F). An undilated eye pupil is shown as 270, a dilated pupil 280 and the limbus of the eye 290. A masked contact lens is shown in FIG. 2(G). Since the patient's eye is supposed to have normal vision without any severe distortions prior to dilation, a wavefront aberrometer measurement may not be necessary. The natural pupil size 270 of the patient before the dilation is first recorded. This may range from 2 to 4 mm in daytime. A masked contact lens 340 is shown in FIG. 2(G). It has substantially a blocking region 320 from the mid to periphery region, and has a clear opening 300 at or near the center at 2 to 4 mm. Without limitation, the central “clear” zone may be tinted to control the level of the light transmission. The size of the central opening may be chosen to match the natural pupil size before dilation. When such masked contact lens is applied to a dilated eye, it will eliminate the problems of quality of vision decrease and light saturation. Such contact lens will control the light rays entering the eye to a level close to that of an undilated eye, and restore the quality of vision to that of an undilated eye.

Furthermore, the masked contact lens may have refractive power in the range of −3 to +3 diopters, built-in in the lens. This is useful for situations wherein certain eyes may have residual refractive error under cycloplegia, when the natural eye lens has lost its ability to accommodate. In this embodiment, an added power will correct for that residual refractive error.

### AN EXAMPLE ILLUSTRATING THE QUALITY OF VISION IMPROVEMENT USING MASKED CONTACT LENSES

A wavefront map of a keratoconus eye with a 6 mm pupil diameter is captured and shown in FIG. 3(A). The cone is shown at the lower left. The point spread function of that eye is shown in FIG. 3(B) having a broad linewidth, which is indicative of poor quality of vision. An image of a letter “F” is shown in FIG. 3(C) without any distortion in the eye, while that same letter “F” is illustrated at FIG. 3(D) as viewed through the keratoconus eye of FIG. 3(A).

A masked contact lens may be generated that blocks incoming light rays except at a circular region of 3 mm opening selected at a region of the wavefront map that is relatively “flat”, in this case near the upper right of the eye map. This is illustrated at FIG. 4(A). We use the square frames in FIGS. 3-5 in reference to the dimension and the location of masked area over the wavefront map. The point spread function of the opening excluding the masked area is shown in FIG. 4 (B), which is improved relative to FIG. 3(B), including a much tighter linewidth. The linewidth shall mean the full width at half maximum, or the width at 85% of the integrated volume under the peak. In either cases, the linewidth is indicative of how narrow is the peak of the PSF. The letter “F” as viewed through this opening, or the eye having a masked contact lens including such opening, that is properly positioned to allow the selected region to be clear is illustrated at FIG. 4(C), showing clear improvement over that in FIG. 3(D).

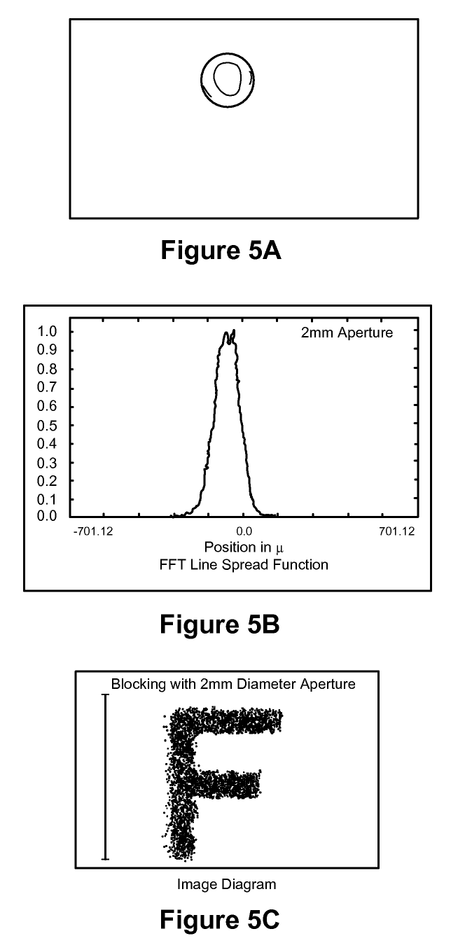

In FIG. 5(A), a further limitation is provided in the clear region of the masked lens of FIG. 4(A). FIG. 5(A) illustrates a limitation to a 2 mm diameter opening region. The corresponding PSF is shown in FIG. 5(B). An improvement in the linewidth tightness is observed. The letter “F” as viewed through such masked cornea region is illustrated in FIG. 5(C).

Even through contact lens is used in our illustrations, a masked lens can be in the form of a contact lens, an intra-corneal lens or an intraocular lens, or a combination thereof. A mask region or regions on any of the lenses mentioned will perform an advantageous function improving the quality of vision. An equivalent masked region can be transferred from a design based on a contact lens to an intra-corneal or intraocular lens.

The present invention is not limited to the embodiments described above herein, which may be amended or modified without departing from the scope of the present invention as set forth in the appended claims, and structural and functional equivalents thereof.

In methods that may be performed according to preferred embodiments herein and that may have been described above and/or claimed below, the operations have been described in selected typographical sequences. However, the sequences have been selected and so ordered for typographical convenience and are not intended to imply any particular order for performing the operations.

In addition, all references cited above herein, in addition to the background and summary of the invention sections, as well as US patent applications nos. 60/826,043, 60/820,340, Ser. Nos. 11/764,160, 11/782,912, 11/746,051 and 11/675,079, and U.S. Pat. Nos. 7,217,375, 6,761,454, 7,234,810, 7,114,808, 6,976,641, 6,761,454, and 6,836,371, also by Dr. Shui Lai, are all hereby incorporated by reference into the detailed description of the preferred embodiments as disclosing alternative embodiments and components.

I claim:

1. A system for reducing wavefront aberrations of an eye, comprising:
(a) a wavefront aberrometer for measuring wavefront information of an eye;
(b) a computer program embodied within one or more storage media for programming one or more processors to derive a mask covering a wavefront aberration portion of the eye based on the measured wavefront information; and
(c) a machine for forming a customized lens including the mask incorporated therein.

2. The system of claim 1, wherein the deriving of the mask comprises an optimization of a figure of merit using a PSF, a MTF, another image quantifier, or combinations thereof.

3. The system of claim 2, wherein the figure of merit comprises a light intensity volume within a circle centered at a PSF peak or at a geometric mean of a PSF intensity distribution.

4. The system of claim 2, wherein the figure or merit uses MTF and area under MTF curve, Nyquist limits, neural response as a function of spatial frequency, best sphero-cylindrical refraction or best calculated eyeglasses prescription, or combinations thereof.

5. The system of claim 1, wherein the customized lens is configured to reduce the wavefront aberrations to make at least one additional line readable in an EDTRS or Snellen eye chart or both.

6. The system of claim 1, wherein the customized lens is configured to reduce the wavefront aberrations to improve contrast sensitivity by at least 0.15 in log scale or 40% in linear scale, or both.

7. The system of claim 1, wherein the machine is configured to write the customized mask accounting for a de-centered position of the lens on the eye.

8. The system of claim 1, wherein the mask is shaped to block distorted rays from one or more distortion regions of the eye, and comprises one or more masked regions from a perimeter of the lens or a ring on the lens or a combination thereof.

9. The system of claim 1, wherein the lens comprises a contact lens, an intra-corneal lens or an intraocular lens, or combinations thereof.

10. A method of reducing aberrations of an eye, comprising:
(a) measuring wavefront aberrations of an eye,
(b) generating a wavefront map or other wavefront information, or both, based on the measuring;
(c) deriving a mask from the wavefront map or other wavefront information, or both; and
(d) forming a customized lens including incorporating therein the mask.

11. The method of claim 10, wherein the deriving of the mask comprises optimizing a figure of merit using a PSF, a MTF, another image quantifier, or combinations thereof.

12. The method of claim 11, wherein the figure of merit comprises a volume of light rays within a circle centered at a PSF peak or at a geometric mean of a PSF intensity distribution.

13. The method of claim 11, wherein the figure or merit uses MTF and an area under MTF curve, Nyquist limits, neural response as a function of spatial frequency, best sphero-cylindrical refraction or best calculated eyeglasses prescription, or combinations thereof.

14. The method of claim 10, comprising reducing the wavefront aberrations to make at least one additional line readable in an EDTRS or Snellen eye chart or both.

15. The method of claim 10, comprising reducing the wavefront aberrations to improve contrast sensitivity by at least 0.15 in log scale or 40% in linear scale, or both.

16. The method of claim 10, comprising writing the mask while accounting for a de-centered position of the lens on the eye.

17. The method of claim 10, comprising shaping the customized mask to block distorted rays from one or more regions of the eye.

18. The method of claim 17, comprising forming one or more masked regions at a perimeter of the lens or a ring on the lens or a combination thereof.

19. A contact lens to improve the quality of vision of an eye after pupil dilation, comprising:
(a) a contact lens; and
(b) a masked region substantially blocking rays from transmitting through the lens and pupil, except at a central region.

20. The contact lens of claim 19, wherein the masked region comprises at least an entire dilated pupil region.

21. The contact lens of claim 19, wherein the central region transmitting light rays comprises an area including at least one dimension between approximately 2 mm and 4 mm in extent.

22. The contact lens of claim 19, wherein the central region comprises a tinted region that partially transmits light rays.

23. The contact lens of claim 22, wherein the tinted region comprises substantially the entire central region.

24. The contact lens of claim 19, wherein the contact lens has a refractive power in a range from −3 to +3 diopters.

25. The contact lens of claim 19, wherein the central region of the mask also includes one or more specific mask portions determined based on a measurement of wavefront aberrations of an eye, and generation based on the measurement of a wavefront map or other wavefront information, or both.
